(19) United States
(12) Patent Application Publication
(10) Pub. No.: US 2007/0015233 A1
(43) Pub. Date: Jan. 18, 2007

(54) Analysis of molecules

(76) Inventor: Francesco Lorenzo Brancia, Manchester, Lancashire (GB)

Correspondence Address: JACOBSON HOLMAN PLLC, 400 SEVENTH STREET N.W., SUITE 600, WASHINGTON, DC, 20004, US

(21) Appl. No.: 10/569,981

(22) PCT Filed: Aug. 17, 2004
(86) PCT No.: PCT/GB04/03535

§ 371 Date: Feb. 28, 2006

(30) Foreign Application Priority Data
Aug. 30, 2003 (GB) 0320415.3

(51) Int. Cl. C12Q 1/37; G01N 33/00

(52) U.S. Cl. 435/23; 436/86

(57) ABSTRACT

There is disclosed a method of analysing one or more molecules having a primary amine or a lysine functionality in two or more samples comprising the steps of: providing a series of reagents having the formula R₁—NH—C(═NH)—OR₂ where R₁ and R₂ are residue groups or atoms, and wherein the chemical formulae of the reagents in the series are identical but each reagent in the series comprises a different combination of isotopes so that reagents in the series are isotopically labelled by way of the molecular mass of each reagent in the series being different to the molecular masses of the other reagents in the series; introducing a different reagent from the series to each sample so as to effect, in each sample, a guanidination reaction between a reagent and moieties having a primary amine or a lysine functionality, thereby producing a plurality of isotopically labelled guanidine or homoarginine derivatives; combining the samples; optionally modifying the isotopically labelled guanidine or homoarginine derivatives to produce further isotopically labelled guanidine or homoarginine derivatives; separating chemically different components of the combined samples whilst substantially retaining together subsets of guanidine or homoarginine derivatives which differ only by virtue of their isotopic labelling; and performing an analysis of one or more subsets of guanidine or homoarginine derivatives which differ only by virtue of their isotopic labelling; in which subsequent to the step of combining the samples the isotopically labelled guanidine or homoarginine derivatives are not selectively bound to a capture reagent.

This invention relates to the analysis of molecules with particular, but by no means exclusive, reference to macromolecules, such as in the analysis of proteins or protein function in complex mixtures, including quantitative analysis.

Current methods of proteome analysis still largely rely on the use of 2 D-PAGE (polyacrylamide gel electrophoresis) for protein purification prior to mass spectrometric (MS) analysis. However, this method has intrinsic limitations. Firstly, the low abundance proteins which represent a large proportion of all gene products synthetised by a living cell remain undetectable within the dynamic range of the staining protocol (Gygi, S. P. et al. “Evaluation of two-dimensional gel electrophoresis-based proteome analysis technology.” Proc. Natl. Acad. Sci. U.S.A. 97.17 (2000): 9390-95). Pre-fractionation of the sample using affinity chromatography (Rigaut, G. et al. “A generic protein purification method for protein complex characterization and proteome exploration.” Nat. Biotechnol. 17.10 (1999): 1030-32), or the utilisation of more sensitive staining methods and larger formats of gel can enhance detection of low-abundance proteins. Secondly, the conditions of a typical 2DGE experiment are denaturing (urea and SDS are chaotropic agents) and, hence, all non-covalent protein-protein interactions are destroyed and with them the possibility of assigning protein function based on interactions with other proteins of known function. In addition, despite attempts at automation, the 2DGEL based approach remains time-consuming and of low-throughput. The shortcomings of 2DGEL have fuelled interests in developing gel-free based separation techniques. In such regard, liquid chromatography may represent an alternative tool to overcome the disadvantages of gel electrophoresis. This task can be accomplished by exploiting the specific properties of the proteins/peptides such as distribution of charge, size, and specific binding affinity. Application of the technique can be combined either with pre-fractionation of the entire proteome in order to reduce its initial complexity (Link, A. J. et al. “Direct analysis of protein complexes using mass spectrometry.” Nat. Biotechnol. 17.7 (1999):676:82), or after proteolytic digestion of protein mixtures obtained through gel (Peng, J and S. P. Gygi. “Proteomics: the move to mixtures.” J. Mass Spectrom. 36.10 (2001): 1083-91). In the latter approach, when mixtures are extremely complex, on-line reversed-phase liquid chromatography (RPLC) is employed to concentrate and separate the peptides produced from tryptic digestion prior to MS analysis. The retention time of each component of the mixture is related to the intrinsic features of each individual peptide that affects its mobility within the chromatographic system. As a consequence, peptides are eluted singularly or in presence of few other components, analysed by MS and isolated prior to tandem mass spectrometric analysis (MS/MS). Interpretation of the mass spectra through the use of database searching engines allows rapid protein identification. To increase speed and quality of data, automatic methods have been developed to replace the manual selection of each precursor ion prior to colisionally induced decomposition (CAD) (Hoyes, E and S. J. Gaskell. “Automatic function switching and its usefulness in peptide and protein analysis using direct infusion microspray quadruple time-of-flight mass spectrometry.” Rapid Commun. Mass Spectrom. 15.19 (2001): 1802-06).

After protein identification, attention focuses on quantification of all components present in the mixture. All mass spectrometry-based quantification methods rely on labelling selectively the sample with different stable isotopes. Isotopes have been introduced by metabolic labelling using labelled nutrients (Pasa, Tolic L. et al. “High Throughput Proteome-Wide Precision Measurements of Protein Expression Using Mass Spectrometry.” J. Am. Chem. Soc. 121 (1999): 7949-50; Pratt, J. M et al. “Stable isotope labelling in vivo as an aid to protein identification in peptide mass fingerprinting.” Proteomics. 2.2 (2002):157-63; Chen, X., L. M. Smith, and E. M. Bradbury. “Site-specific mass tagging with stable isotopes in proteins for accurate and efficient protein identification 13.” Anal. Chem. 72.6 (2000): 1134-43; Ong, S. E., I. Kratchmarova, and M. Mann. “Properties of 13C-substituted arginine in stable isotope labelling by amino acids in cell culture (SILAC) 1.” J. Proteome. Res. 2.2 (2003): 173-81), or by chemical derivatisation of the peptide functional groups (Gygi, S. P. et al. “Quantitative analysis of complex protein mixtures using isotope-coded affinity tags.” Nat. Biotechnol. 17.10 (1999) 994-99; Goodlett, D. R. et al. “Differential stable isotope labelling of peptides for quantitation and de novo sequence derivation.” Rapid Commun. Mass Spectrom. 15.14 (2001): 1214-21; Munchbach, M. et al. “Quantitation and facilitated de novo sequencing of proteins by isotopic N-terminal labelling of peptides with a fragmentation-directing moiety.” Anal. Chem. 72.17 (2000): 4047-57) or enzymatic labelling through incorporation of ¹⁸O (Yao, X. et al. “Proteolytic 18O labelling for comparative proteomics: model studies with two serotypes of adenovirus 1.” Anal. Chem. 73.13 (2001): 2836-42). The common strategy relies on parallel derivatisation of each set of proteins/peptides with isotopic isomers of the same chemical reagent/label. The samples are then pooled and quantification is achieved by using mass spectrometry after purification. Different moieties within the molecules can be targeted by the labelling agent: for example, cysteine thiol groups (Gygi, S. P. et al. “Quantitative analysis of complex protein mixtures using isotope-coded affinity tags.” Nat. Biotechnol. 17.10 (1999): 994-99, the contents of which are hereby incorporated herein by reference; International Publication WO 00/11208, the contents of which are hereby incorporated herein by reference; Qiu, Y. et al. “Acid-labile isotope-coded extractants: a class of reagents for quantitative mass spectrometric analysis of complex protein mixtures 1.” Anal. Chem. 74.19 (2002) 4969-79), tryptophan (Kurama, H. et al. “An approach to quantitative proteome analysis by labelling tryptophan residues.” Rapid Commun. Mass Spectrom. 17.14 (2003): 1642-50), phosphate groups (Goshe, M. B. et al. “Phosphoprotein isotope-coded affinity tag approach for isolating and quantitating phosphopeptides in proteome-wide analyses.” Anal. Chem. 73.11 (2001): 2578-86) and primary amines (Liu, P. and Reigner, F. E. “An isotope coding strategy for proteomics involving both amine and carboxyl group labelling 1.” J Prot. Research 1.5 (2002): 443-50; Goodlett, D. R. et al. “Differential stable isotope labelling of peptides for quantitation and de novo sequence derivation.” Rapid Commun. Mass Spectrom. 15.14 (2001): 1214-21) have been targeted.

Of all amino acid residues lysine plays a crucial role in affecting the final appearance of a MS spectrum. Krause et al noted that lysine containing peptides generated by tryptic digestion of proteins produce MALDI ions detected in lesser abundance than those incorporating arginine (Krause, E., H. Wenschuh, and P. R. Jungblut. “The dominance of arginine-containing peptides in MALDI-derived tryptic mass fingerprints of proteins.” Anal. Chem. 71.19 (1999): 4160-65). Modification of the gas-phase basicity through selective conversion of the lysine into homoarginine (a process known as guanidination) shows a beneficial effect on detection of peptide ions under MALDI analysis (Brancia, F. L., S. G. Oliver, and S. J. Gaskell. “Improved matrix-assisted laser desorption/ionization mass spectrometric analysis of tryptic hydrolysates of proteins following guanidination of lysine-containing peptides.” Rapid Commun. Mass Spectrom. 14.21 (2000): 2070-73; Beardsley, R. L., J. A. Karty, and J. P. Reilly. “Enhancing the intensities of lysine-terminated tryptic peptide ions in matrix-assisted laser desorption/ionization mass spectrometry.” Rapid. Commun. Mass Spectrom. 14.23 (2000): 2147-53). A discussion of the nomenclature surrounding the guanidination reaction can be found in J. Peptide Sci 8: 285-287 (2002).

Emili and Cagney have proposed a strategy for proteome analysis based on differential guanidination of C-terminal lysine residues of tryptic peptides followed by capillary liquid chromatography-electrospray tandem mass spectrometry (Cagney, G. and A. Emili. “De novo peptide sequencing and quantitative profiling of complex protein mixtures using mass-coded abundance tagging.” Nat. Biotechnol. 20.2 (2002): 163-70, the contents of which are hereby incorporated herein by reference). Although the method, termed mass-coded abundance tagging (MCAT), facilitates interpretation of product ion spectra, the determination of protein expression based on the electrospray response of treated and untreated samples suffers due to the intrinsic differences in ionisation efficiency existing between lysine and homoarginine containing peptides. It has been demonstrated that ionisation behaviour of homoarginine terminal peptides is in accordance with the postulated higher stability of the guanidino group with respect to the amino group of lysine side chains (Brancia, F. L., M. E. Openshaw, and S. Kumashiro. “Investigation of the electrospray response of lysine-, arginine-, and homoarginine-terminal peptide mixtures by liquid chromatography/mass spectrometry 1.” Rapid Commun. Mass Spectrom. 16.24 (2002): 2255-59).

Thus, it will be apparent that a need exists for an improved method of proteome analysis directed towards the recognition of lysine containing proteins and peptides. The present invention satisfies this need.

According to the invention there is provided a method of analysing one or more molecules having a primary amine or a lysine functionality in two or more samples comprising the steps of:

providing a series of reagents having the formula R₁—NH—C(═NH)—OR₂ where R₁ and R₂ are residue groups or atoms, and wherein the chemical formulae of the reagents in the series are identical but each reagent in the series comprises a different combination of isotopes so that reagents in the series are isotopically labelled by way of the molecular mass of each reagent in the series being different to the molecular masses of the other reagents in the series;

introducing a different reagent from the series to each sample so as to effect, in each sample, a guanidination reaction between a reagent and moieties having a primary amine or a lysine functionality, thereby producing a plurality of isotopically labelled guanidine or homoarginine derivatives;

combining the samples;

optionally modifying the isotopically labelled guanidine or homoarginine derivatives to produce further isotopically labelled guanidine or homoarginine derivatives;

separating chemically different components of the combined samples whilst substantially retaining together subsets of guanidine or homoarginine derivatives which differ only by virtue of their isotopic labelling;

and performing an analysis of one or more subsets of guanidine or homoarginine derivatives which differ only by virtue of their isotopic labelling;

in which subsequent to the step of combining the samples the isotopically labelled guanidine or homoarginine derivatives are not selectively bound to a capture reagent.

In some embodiments of the invention the molecule is a primary amine, in which instance the guanidination reaction produces a guanidine derivative by the conversion of —NH₂ to —NH—C(═NH)NH—R₁. Advantageously, the molecule is a drug having a primary amine functionality, and the guanidination reaction produces a plurality of isotopically labelled guanidine derivatives. Relatively small molecules having primary amine functionalities can be analysed.

According to a preferred aspect of the invention there is provided a method of analysing one or more macromolecules having a lysine functionality in two or more samples comprising the steps of:

providing a series of reagents having the formula R₁—NH—C(—NH)—OR₂ where R₁ and R₂ are residue groups or atoms, and wherein the chemical formulae of the reagents in the series are identical but each reagent in the series comprises a different combination of isotopes so that reagents in the series are isotopically labelled by way of the molecular mass of each reagent in the series being different to the molecular masses of the other reagents in the series;

introducing a different reagent from the series to each sample so as to effect, in each sample, a guanidination reaction between a reagent and moieties having a lysine functionality, thereby producing a plurality of isotopically labelled, homoarginine derivatives;

combining the samples;

optionally modifying the isotopically labelled homoarginine derivatives to produce further isotopically labelled homoarginine derivatives;

separating chemically different components of the combined samples whilst substantially retaining together subsets of homoarginine derivatives which differ only by virtue of their isotopic labelling;

and performing an analysis of one or more subsets of homoarginine derivatives which differ only by virtue of their isotopic labelling;

in which subsequent to the step of combining the samples the isotopically labelled homoarginine derivatives are not selectively bound to a capture reagent.

In this way, the problems associated with the technique of Emili and Cagney, ie, that treated and untreated samples exhibit different ionisation efficiencies are overcome. This is because all of the samples used in the analysis have undergone a guanidination reaction, and thus in all samples lysine containing moieties are converted into homoarginine containing moieties. Furthermore, conversion of lysine containing moieties into homoarginine containing moieties results in an increase in ionisation efficiencies for each of the samples. Furtherstill, guanidination increases the overall number of fragments usable for database searching. In comparison to the so-called ICAT (isotope coded affinity tag) technique of Gygi et al and WO 00/11208, the present invention has the advantage of being specific to lysine rather than cysteine (the abundance of cysteine amino acid resides in proteins being relatively low). Furthermore, the present invention does not utilise a capture step involving an affinity tag, and thus a level of complexity is removed.

The macromolecules may comprise one or more proteins, protein functions and/or peptides having a lysine functionality.

The method may comprise the step of converting proteins into peptides. The step of modifying the isotopically labelled homoarginine derivatives may comprise converting proteins present in the isotopically labelled homoarginine derivatives into peptides. However, it is possible to convert proteins in the samples into peptides prior to treatment with the reagent. The conversion (of homoarginine derivatives and/or proteins in the samples prior to treatment with the reagent) might be performed enzymatically. Alternatively, it is possible to analyse the direct product of the guanidination reaction between the reagent and a protein “directly”, ie, without digestion or fragmentation of the protein into peptides (top-down approach).

Relative abundances of macromolecules in the two or more samples may be determined, and the step of performing an analysis may comprise measuring the relative abundances of a subset of homoarginine derivatives which differ only by virtue of their isotopic labelling and equating the measured relative abundances with the relative abundances in the two or more samples of the macromolecule from which the subset of homoarginine derivatives originated. In this way, relative expression levels of proteins in the two or more samples may be determined by equating the measured relative abundances of a subset of homoarginine derivatives with relative expression levels of the protein from which the subset of homoarginine derivatives originated.

Macromolecules may be identified by the analysis of the subsets of homoarginine derivatives. Advantageously, proteins, protein function and/or peptides may be identified by the analysis of the subsets of homoarginine derivatives.

The analysis may comprise the step of comparing data generated by an analytical technique with sequence data. Alternatively, the analysis may comprise a de novo analysis.

The analysis may comprise mass spectrometric analysis. Mass spectrometric analysis can rely on MS techniques such as electrospray, matrix assisted laser desorption ionisation (MALDI), chemical reaction interface mass spectrometry (CRIMS), atmospheric pressure chemical ionisation (APCI), electron impact (EI), and fast atomic bombardment (FAB). The mass spectrometric analysis may comprise tandem mass spectrometry. Techniques such as collision induced decomposition (CID), electron capture decomposition (ECD), surface induced decomposition (SID), and infrared multiphoton decomposition (IRMPD) may be used.

The step of separating chemically different components of the combined samples may comprise utilising a chromatographic separation system. The chromatographic separation system may utilise liquid chromatography.

The chromatographic separation system may utilise gas chromatography.

Gel separation techniques might be employed, but are less preferred.

R₂ may be a moiety that is compatible with the guanidination reaction. R₂ may be an alkyl group, which may be CH₃, C₂H₅ or C₃H₇.

R₁ may be H, or COR₃, where R₃ is an alkyl group, such as CH₃, C₂, H₅. C₃H₇ or C₄H₉.

Methods in accordance with the invention will now be described with reference to the accompanying drawings in which:

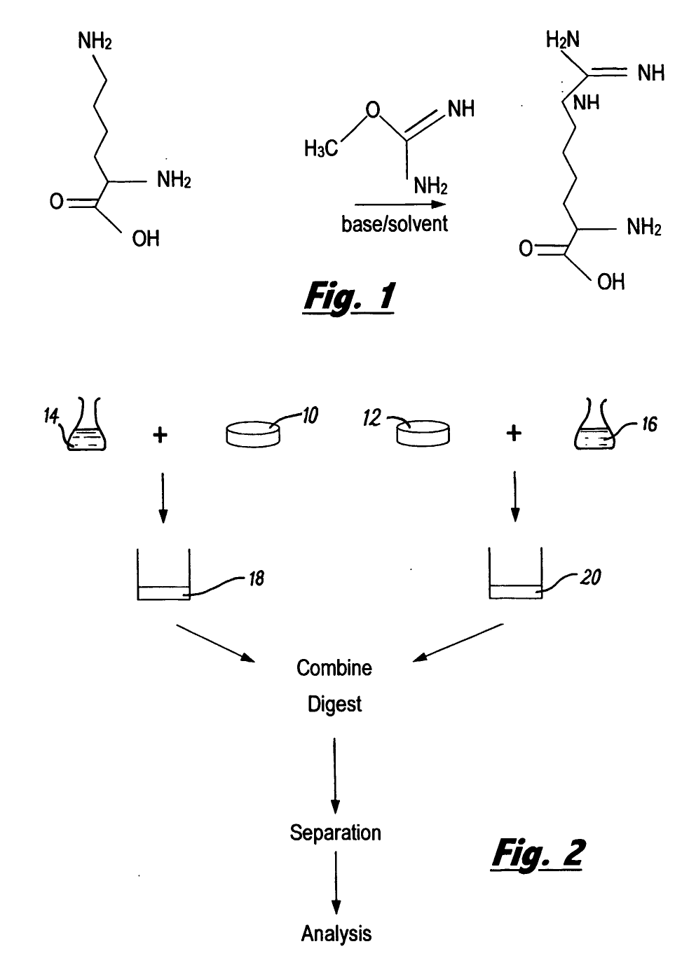

FIG. 1 shows the guanidination of lysine into homoarginine;

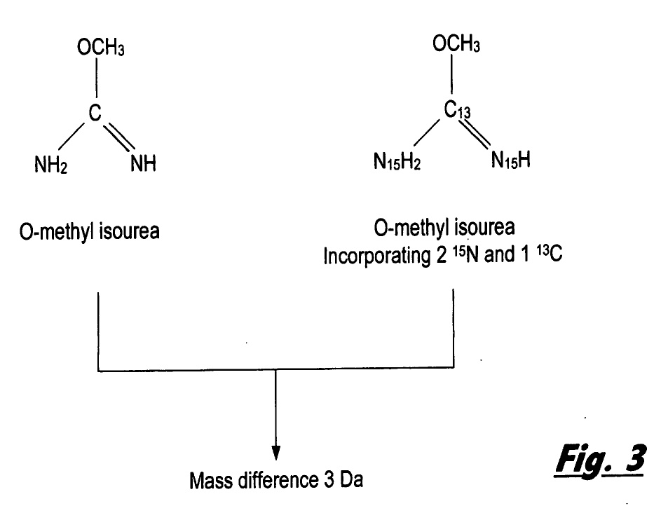

FIG. 2 shows a scheme for quantifying differential protein expression;

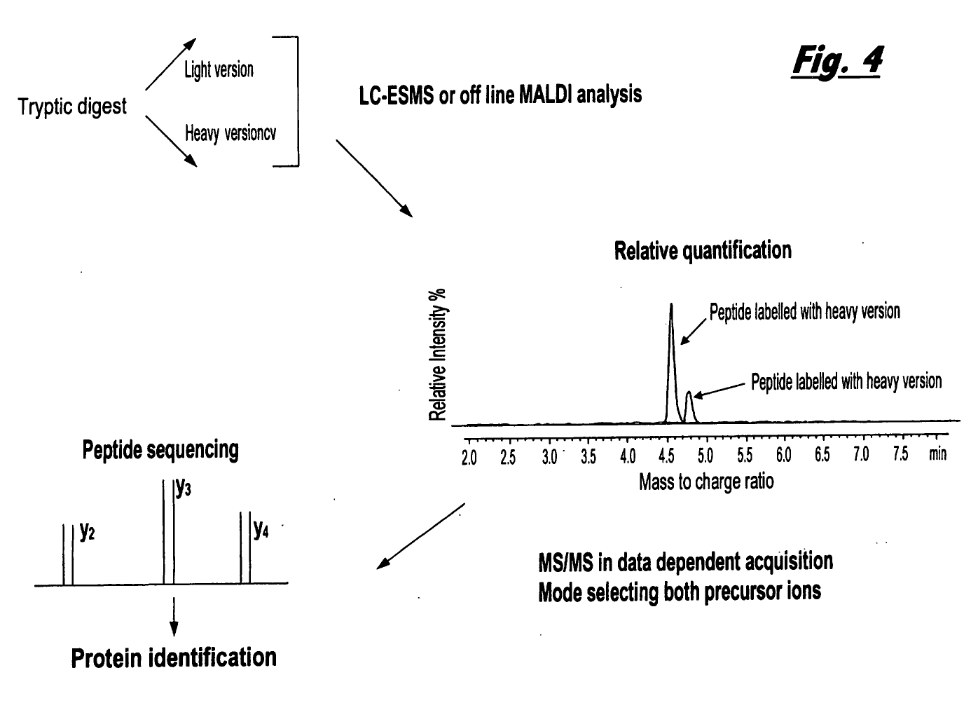

FIG. 3 shows O-methylisourea and its isotopic isomer containing three different isotopes; and

FIG. 4 shows the general strategy of a method of the invention.

The present invention exploits the guanidination reaction to inter alia produce improvements to the techniques of Gygi et al, ibid (Nat. Biotechnol. 17.10 (1999): 994-99), WO 00/11208, and Emili and Cagney.

FIG. 1 depicts the guanidination of lysine, typically at around pH 10 or greater, into homoarginine using O-methyl isourea. In a general sense, this aspect of the invention provides methods for analysing macromolecules having a lysine functionality. A macromolecule can be analysed by a suitable technique such as mass spectrometry or a spectroscopic method. In important embodiments the invention provides analytical methods for the separation, purification and identification of proteins and peptides in mixtures of proteins and peptides. The method employs reagents which exploit the reactivity between lysine amino groups and isourea derivatives to separate, purify and determine peptides and proteins present in a mixture. Homoarginine derivatives are created which isolate lysine containing proteins and peptides In other embodiments, molecules having a primary amine functionality are analysed using the guanidination reaction to produce guanidine derivatives having a —NH—C(═NH)NH—R₁ functionality.

The chemistry between a lysine side chain amino group and isourea moiety is well established. In the majority of cases, the presence of the N-terminal amino group does not interfere with the reaction occurring on the lysine amino groups.

An additional advantage associated with the use of guanidination in peptide sequencing stems from the predictable addition of a guanidino moiety into the peptide structure. Selective modification of the E-lysine amino group produces a shift of 42 Da which affects solely ions with the proton located at C-terminus (x, y, z). Comparison of the ion signals deriving from the two product ion spectra enable assessment of the type of ion encountered.

FIG. 2 shows a scheme for quantifying differential protein expression. Two protein mixtures 10, 12 which represent different cell states are treated with chemically identical, isotopically different reagents 14, 16. Thus, a first protein mixture 10 may be treated with an isotopically “light” reagent 14, and a second protein mixture 12 may be treated with an isotopically “heavy” reagent 16. Guanidination produces a mixture of homoarginine derivates 18, which, by virtue of the reagents utilised, is “light” and a mixture of homoarginine derivatives 20 which, by virtue of the reagents utilised, is “heavy”. The mixtures 18, 20 are combined, and proteins (including those forming part of the homoarginine derivatives) are digested to peptides using techniques which are well known in the art. The homoarginine derivatives (now comprising peptides) are separated using a separation technique, such as a chromatographic technique. Liquid chromatography (LC) is a preferred technique, and variants such as capillary liquid chromatography (iLC) can be used. Separated homoarginine derivatives can be analysed using mass spectrometry (MS).

Identical peptides emanating from the mixtures 10, 12 give rise to chemically identical homoarginine derivatives which more or less coelute from the LC. However, due to the “light” and “heavy” nature of the binding reagents, these homoarginine derivatives have different molecular masses which are identifiable in a mass spectrum at different values of mass-to-charge ratio. Thus, relative quantification of protein expression is possible by comparing the relative abundances of the ion peaks corresponding to the “light” and “heavy” homoarginine derivatives. MS/MS analysis of the elutants provides sequence information which enables identification of the protein through computer searching of the experimentally obtained sequence information against databases. Further details of suitable analysis schemes can be found in WO 00/11208 and Gygi et al.

In further embodiments of the invention, calibrations are performed using samples containing known concentrations of proteins, in order to obtain a quantitative relationship between peptide signal obtained during analysis and the absolute amount of protein present in the sample.

Important embodiments of the invention combine the use of isotopic labelled molecules based on O-methylisourea with chromatographic separation prior to MS analysis. The method provides protein identification via database searching or via de novo interpretation together with quantification based on the abundances of ions labelled with the chemical tags containing different isotopes. For instance, as shown in FIG. 3, O-methylisourea and its isotopic isomer containing two ¹⁵N and one ¹³C could be the reagents used. Isotopic variants can be employed based on the following formula: R₁—NH—C(═NH) —OR₂ where R₁═H, CO—R₃, and R₂ is alkyl. R₃ an be an alkyl group, such as methyl. Two mixtures containing the same proteins are differentially labelled with the reagents previously described. The sample can be labelled before or after proteolytic digestion. The samples are combined and separated by liquid chromatography. Different types of interactions can be exploited in the choice of stationary phase and reverse phase. Peptides only differing for the isotopes incorporated in the chemical tag are eluted together. FIG. 4 depicts the strategy employed. Concomitant mass spectrometric analysis of both ions allows determination of their abundances and therefore quantification can be achieved. Subsequent isolation of the ion species permits tandem mass analysing using different means, such as collision induced decomposition (CID) electron capture decomposition (ECD), surface induced decomposition (SID), infrared multiphoton decomposition (IRMPD) etc. By comparing the product ion spectra (MS/MS), it is possible to determine the type of fragment ion. Considering the variants of O-methylisourea shown in FIG. 3, the x, y, z ions bearing the portion on the part of molecule, on which the derivatisation occurs, will be shifted of 3 Da. Conversely, the m/z values of a, b, c ions comprising the fragment ions which retain the proton at N-terminus are unmodified. The method can be amenable of automation and software based on these characteristics can be developed.

Mass spectrometric analysis can rely on MS techniques such as electrospray, matrix assisted laser desorption ionisation (MALDI), chemical reaction interface mass spectrometry (CRIMS), atmospheric pressure chemical ionisation (APCI), electron impact (EI), and fast atomic bombardment (FAB).

Preparative Methods

Formation of O-methylisourea Hydrogensulfate

Urea (20 g, 0.33 mol) was added portionwise over one hour to a stirred solution of dimethyl sulfate (30.1 ml, 0.33 mol) at 110-115° C. After stirring for two hours at 110° C., the reaction was cooled to room temperature. Concentrated sulfuric acid (18.5 ml, 0.33 mol) was cautiously dissolved in ether (171 ml) with cooling. This was then added to the oil of the reaction along with acetone (171 ml).

Synthesis of N-acetyl-O-methylisourea Adduct.

A suspension of O-methylisourea sulfate (1.23 g, 10 mmol) in acetic anhydride (20 ml) and pyridine (2.4 ml, 30 mmol) was stirred overnight at 30° C. under N₂. The volatiles were then evaporated and methanol was added. A white solid was only partially soluble and was removed by filtration and analysed.

Formation of ¹⁵N₂ labelled O-methylisourea Hydrogensulfate

Urea (¹⁵N₂ labelled) (4 g, 0.064 mol) was added portionwise over 15 minutes to a stirred solution of dimethyl sulfate (5.8 ml, 0.065 mol) at 114-115° C. After stirring for 1.5 hours at 110-115° C., the reaction was cooled to room temperature. Concentrated sulfuric acid (3.6 ml, 0.064 mol) was cautiously dissolved in ether (33 ml) with cooling, and this was then added to the oil of the reaction along with acetone (33 ml).

Formation of ¹⁵N₂ Labelled O-methylisourea Sulfate

An opaque solution of barium hydroxide monohydrate (1.94 g, 0.01 mol) in water (10.6 ml) at 60° C. was added to a stirred cooled solution of ¹⁵N₂ labelled O-methylisourea sulfate (3.52 g, 0.021 mol) in water (˜30 ml) and a milky precipitate was formed. This was centrifuged at 2500 rpm and the supernatant was decanted into another centrifuge tube and spun again at 2500 rpm for five minutes to ensure that no solid material was present. The colourless solution was then decanted and evaporated to dryness. Acetone was added and the product crystallised to give the product as a white solid, yield 2.07 g (82%). Melting point 159.0-159.5° C.

Formation of ¹⁵N₂ labelled N-trideuterioacetyl-O-methylisourea

A suspension of ¹⁵N₂ labelled O-methylisourea sulfate (0.818 g, 6.5 mmol) in acetic anhydride d6 (13.1 ml, 139 mmol) and pyridine (0.53 ml, 6.5 mmol) was stirred overnight at 27° C. The volatiles were then evaporated at T≦30° C. and co-evaporated with toluene. Anhydrous methanol (˜8 ml) was added and the material swirled. A portion of the material did not dissolve and was filtered to give 0.329 g of a white powder which was identified as target compound. Ether was then added to the filtrate and a precipitate formed which was filtered, dried and then washed with anhydrous methanol (˜4 ml) to give a further 0.01 g of pure product. Total yield 0.400 g (51%).

Synthesis of O-methylisourea Adduct of Valeric Acid

A suspension of valeric acid (0.22 ml, 2 mmol), O-methylisourea sulfate (246 g, 2 mmol) and EDC (0.77 g, 4 mmol) in diisopropylethulamine (0.36 ml, 24 mmol) and DMF (20 ml) was stirred overnight. The reaction mixture was then evaporated using a rotary evaporator with a bath temperature of 35° C. and a high-vacuum pump. After evaporation, the crude product was dissolved in a mixture of DCM, methanol and acetone and added to a silica gel column containing DCM. The desired product eluted in the first three fractions and was fairly pure (as demonstrated by nmr spectroscopy). An attempt was made to re-purify this material by column chromatography (eluent=DCM/methanol) but no material corresponding to the product was isolated.

The reaction was repeated on a larger scale: a suspension of valeric acid (0.44 ml, 4 mmol), O-methylisourea sulfate (493 mg, 4 mmol) and EDC (1.53 g, 8 mmol) in diisopropylethylamine (0.72 ml, 4 mmol) and DMG (150 ml) was stirred overnight. The reaction mixture was then evaporated using a rotary evaporator with a bath temperature of 35° C. and a high-vacuum pump. After evaporation, dioxane was added and the reaction mixture was filtered, thus removing unreacted O-methylisourea sulfate. The dioxane soluble material was purified by silica gel column chromatography using dioxane as eluent. The product was obtained as an off-white solid 0.27 g (42%) and was not as pure as the material previously isolated.

1. A method of analysing one or more molecules having a primary amine or a lysine functionality in two or more samples comprising the steps of:
providing a series of reagents having the formula R₁—NH—C(═NH)—OR₂ where R₁ and R₂ are residue groups or atoms, and wherein the chemical formulae of the reagents in the series are identical but each reagent in the series comprises a different combination of isotopes so that reagents in the series are isotopically labelled by way of the molecular mass of each reagent in the series being different to the molecular masses of the other reagents in the series;
introducing a different reagent from the series to each sample so as to effect, in each sample, a guanidination reaction between a reagent and moieties having a primary amine or a lysine functionality, thereby producing a plurality of isotopically labelled guanidine or homoarginine derivatives;
combining the samples;
optionally modifying the isotopically labelled guanidine or homoarginine derivatives to produce further isotopically labelled guanidine or homoarginine derivatives;
separating chemically different components of the combined samples whilst substantially retaining together subsets of guanidine or homoarginine derivatives which differ only by virtue of their isotopic labelling;
and performing an analysis of one or more subsets of guanidine or homoarginine derivatives which differ only by virtue of their isotopic labelling;
in which subsequent to the step of combining the samples the isotopically labelled guanidine or homoarginine derivatives are not selectively bound to a capture reagent.

2. A method according to claim 1 in which the molecule is a drug having a primary amine functionality, and the guanidination reaction produces a plurality of isotopically labelled guanidine derivatives.

3. A method according to claim 1 in which the molecule is a macromolecule having a lysine functionality, and the guandination reaction produces a plurality of isotopically labelled homoarginine derivatives.

4. A method according to claim 3 in which the macromolecules comprise one or more proteins, protein functions and/or peptides having a lysine functionality.

5. A method according to claim 4 comprising the step of converting proteins into peptides.

6. A method according to claim 5 in which the step of modifying the isotopically labelled homoarginine derivatives comprises converting proteins present in the isotopically labelled homoarginine derivatives into peptides.

7. A method according to claim 3 in which relative abundances of macromolecules in the two or more samples are determined, and wherein the step of performing an analysis comprises measuring the relative abundances of a subset of homoarginine derivatives which differ only by virtue of their isotopic labelling and equating the measured relative abundances with the relative abundances in the two or more samples of the macromolecule from which the subset of homoarginine derivatives originated.

8. A method according to claim 7 wherein said macromolecules are proteins, protein functions and/or peptides having a lysine functionality, in in which relative expression levels of proteins in the two or more samples are determined by equating the measured relative abundances of a subset of homoarginine derivatives with relative expression levels of the protein from which the subset of homoarginine derivatives originated.

9. A method according to claim 3 in which macromolecules are identified by the analysis of the subsets of homoarginine derivatives.

10. A method according to claim 4, wherein said macromolecules are proteins, protein functions and/or peptides having a lysine functionality, in which proteins, protein function and/or peptides are identified by the analysis of the subsets of homoarginine derivatives.

11. A method according to claim 1 in which the analysis comprises the step of comparing data generated by an analytical technique with sequence data.

12. A method according to claim 1 in which the analysis comprises mass spectrometric analysis.

13. A method according to claim 12 in which the mass spectrometric analysis comprises tandem mass spectrometry.

14. A method according to claim 1 in which the step of separating chemically different components of the combined samples comprises utilising a chromatographic separation system.

15. A method according to claim 14 in which the chromatographic separation system utilises liquid chromatography.

16. A method according to claim 14 in which the chromatographic separation system utilises gas chromatography.

17. A method according to claim 1 in which R₂ is an alkyl group.

18. A method according to claim 17 in which R₂ is CH₃.

19. A method according to claim 1 in which R₁ is H.

20. A method according to claim 5 in which R₁ is COR₃, where R₃ is an alkyl group.
